(19) United States
(12) Patent Application Publication
(10) Pub. No.: US 2026/0090786 A1
(43) Pub. Date: Apr. 2, 2026

(54) MACHINE LEARNING BASED CLINICAL SCAN GUIDANCE ALGORITHM FOR ABDOMINAL ULTRASOUND

(71) Applicant: GE Precision Healthcare LLC, Waukesha, WI (US)

(72) Inventors: Hiroshi Hashimoto, Hino (JP); Yi Hou, Hino (JP)

(21) Appl. No.: 19/347,259

(22) Filed: Oct. 1, 2025

(30) Foreign Application Priority Data
Oct. 2, 2024 (JP) 2024-173112

(51) Int. Cl. A61B 8/00; G06T 7/70; G16H 30/40

(52) U.S. Cl. CPC A61B 8/4245; A61B 8/463; G06T 7/70; G16H 30/40; G06T 2207/30004

(57) ABSTRACT

A first ultrasound image of an ultrasound probe at a first time point is analyzed to identify a predetermined structure within an imaging subject and to identify a first position thereof. In addition, a first area indicator corresponding to a first area of the predetermined structure in the first ultrasound image is output. The system outputs guide information instructing the operator to move the ultrasound probe to a position where an ultrasound image can be obtained in which the predetermined structure is displayed more favorably. A second area indicator having an attribute corresponding to a second area of the predetermined structure in a second ultrasound image at a second time point is also output in a manner that allows recognition of a change over time relative to the first area indicator.

This application claims priority to Japanese Patent Application No. 2024-173112, which was file on October 2, 2024 at the Japanese Patent Office. The entire contents of the above-listed application are incorporated by reference herein their entirety.

### TECHNICAL FIELD

The present invention relates to a program for guiding an operation of an ultrasound probe, and more particularly, to a method for the structure of a target displayed on an ultrasound image obtained by using an ultrasound probe favorably.

### BACKGROUND

When performing an ultrasonic examination, an operator can dispose an ultrasonic probe at any position on a scan target, orient the probe in any direction, perform imaging, and obtain a non-destructive/non-invasive ultrasonic image.

On the other hand, for example, in a case where it is necessary to perform an ultrasound examination on the entire target site evenly for the purpose of a health checkup or the like, the operator may be required to dispose the ultrasound probe at a predetermined position and direction. For example, the Japan Gastroenterological Endoscopy Society has defined 25 types of recommended recording cross sections (ultrasound B-mode images), and for each cross section, the position and direction in which the operator places the ultrasound probe on the subject is designated. An image recorded according to such a procedure is used as evidence of thorough examination and for reporting the presence or absence of a detected abnormality. At this time, it is required to draw the organ included in each cross-section at the center of the screen and in the maximum divided surface as much as possible and record the image.

However, there are cases where appropriate image recording cannot be performed due to a difference in technical skill of the operator, fatigue caused by repeating the examination on a large number of people, or the like.

### SUMMARY

Therefore, a system that supports an operator of an ultrasound probe to perform an appropriate operation in an easily understandable manner in order to obtain a preferable ultrasound image is desired.

According to a first aspect of the present disclosure, an ultrasound image generation system for displaying an ultrasound image on a display device is provided. The ultrasound image generation system includes a processor and a non-transitory storage medium for storing a program. The program is configured to cause the processor to execute: analyzing a first ultrasound image of an ultrasound probe at a first time point to identify a predetermined structure within an imaging subject; identifying a first position in the first ultrasound image of the predetermined structure; outputting a first area indicator having an attribute corresponding to a first area in the first ultrasound image of the predetermined structure; outputting guide information for instructing an operator who operates an ultrasound probe to move the ultrasound probe to a position where an ultrasound image in which the predetermined structure is displayed more favorably can be obtained, based on the specified first position; and a step for outputting a second area indicator having an attribute corresponding to a second area in a second ultrasound image at a second time point of the predetermined structure in a manner that a change over time with respect to the first area indicator is recognizable.

In a second aspect of the present disclosure, a program for displaying an ultrasound image on a display device of an ultrasound image generation system is provided. The program is configured to cause a processor to execute: analyzing a first ultrasound image of an ultrasound probe at a first time to identify a predetermined structure within an imaging subject; identifying a first position in the first ultrasound image of the predetermined structure; outputting a first area indicator having an attribute corresponding to a first area in the first ultrasound image of the predetermined structure; outputting guide information for instructing an operator who operates an ultrasound probe to move the ultrasound probe to a position where an ultrasound image in which the predetermined structure is displayed more favorably can be obtained, based on the specified first position; and outputting a second area indicator having an attribute corresponding to a second area in a second ultrasound image at a second time point of the predetermined structure in a manner that a change over time with respect to the first area indicator is recognizable.

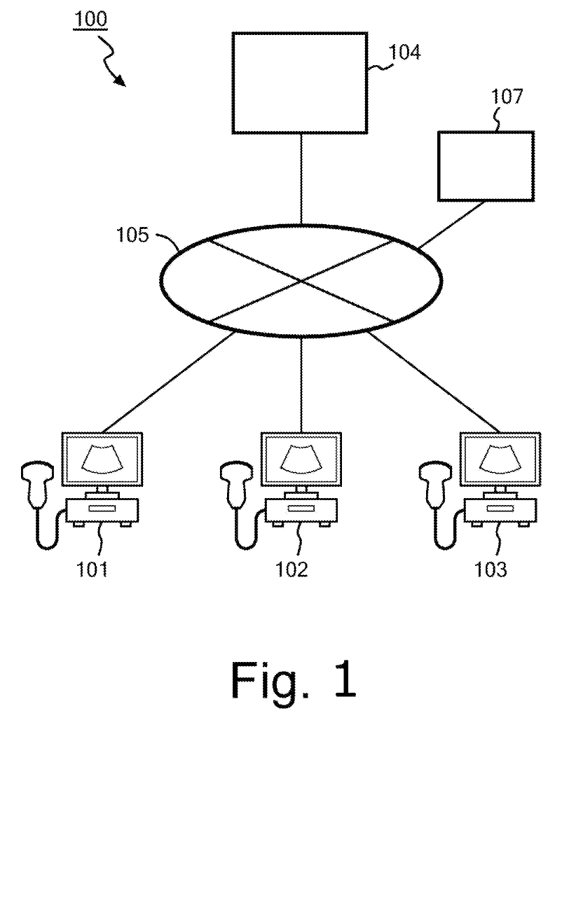

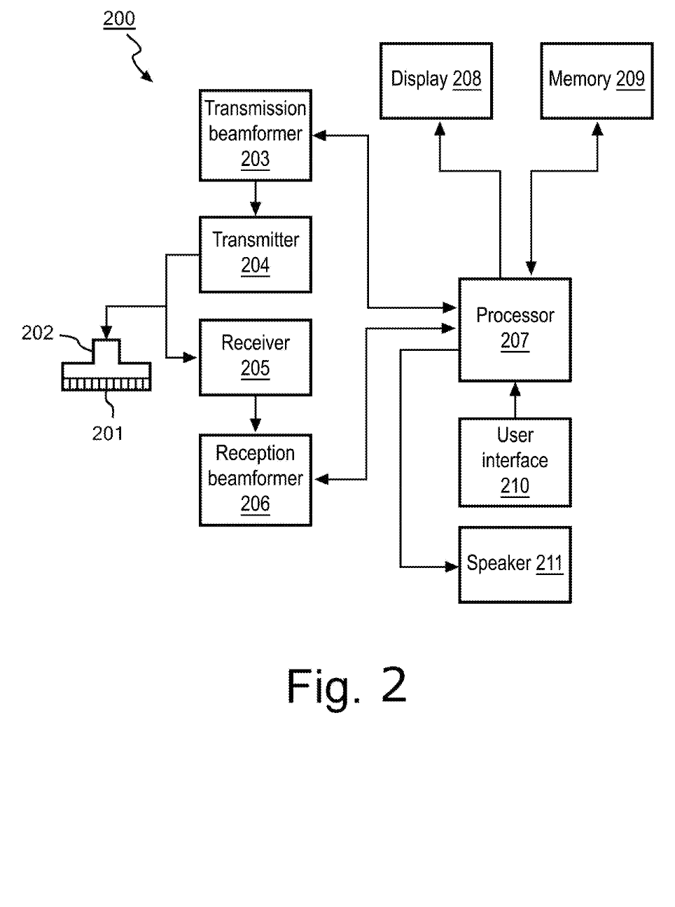

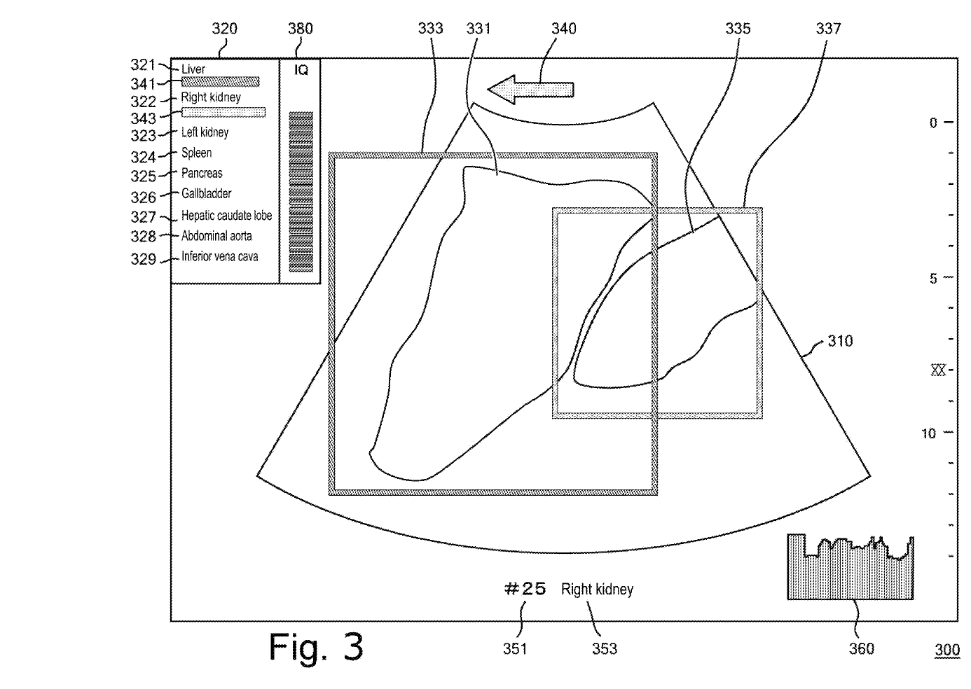

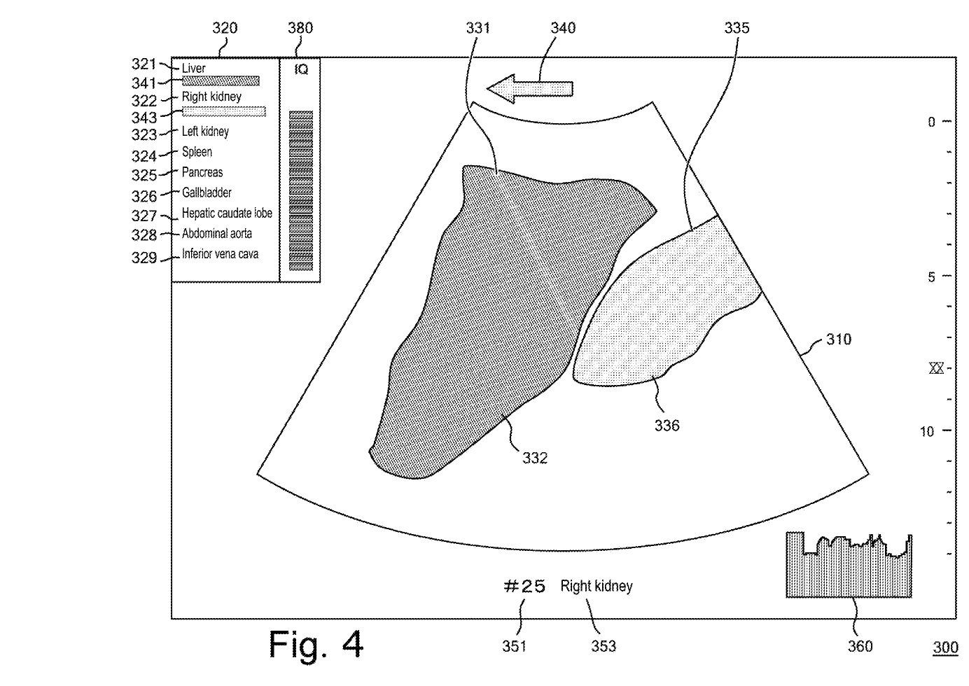

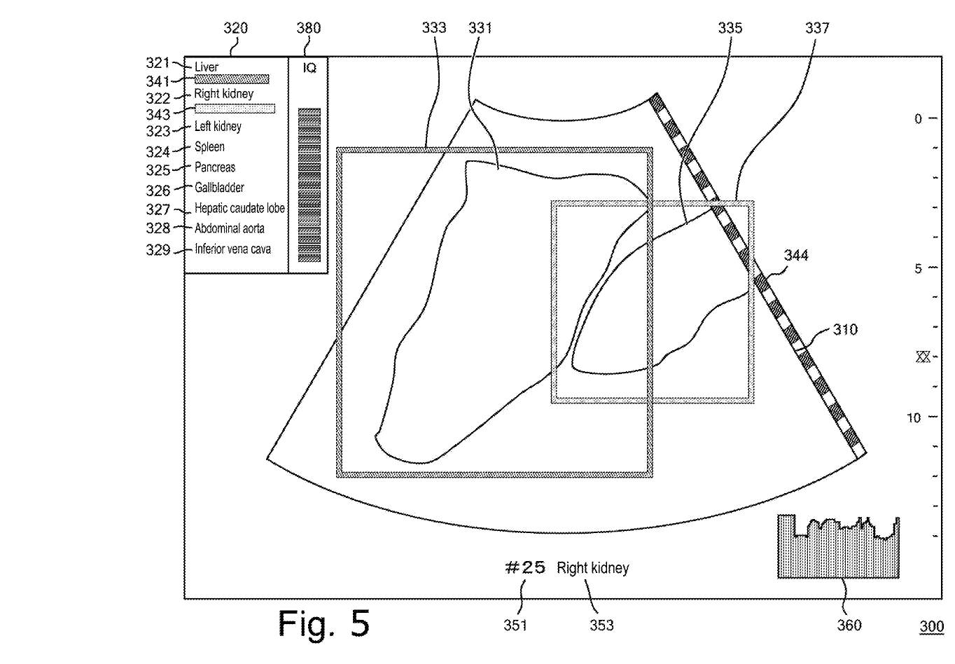

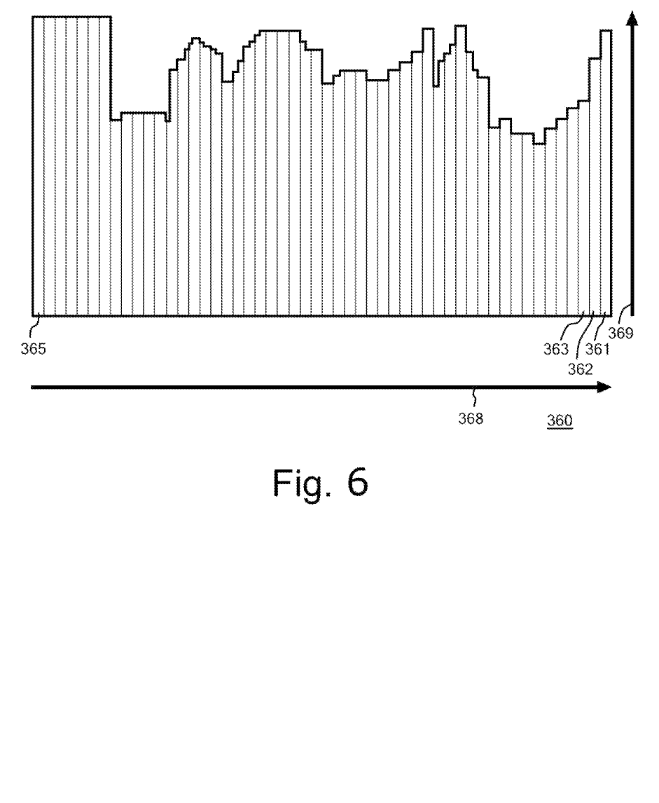

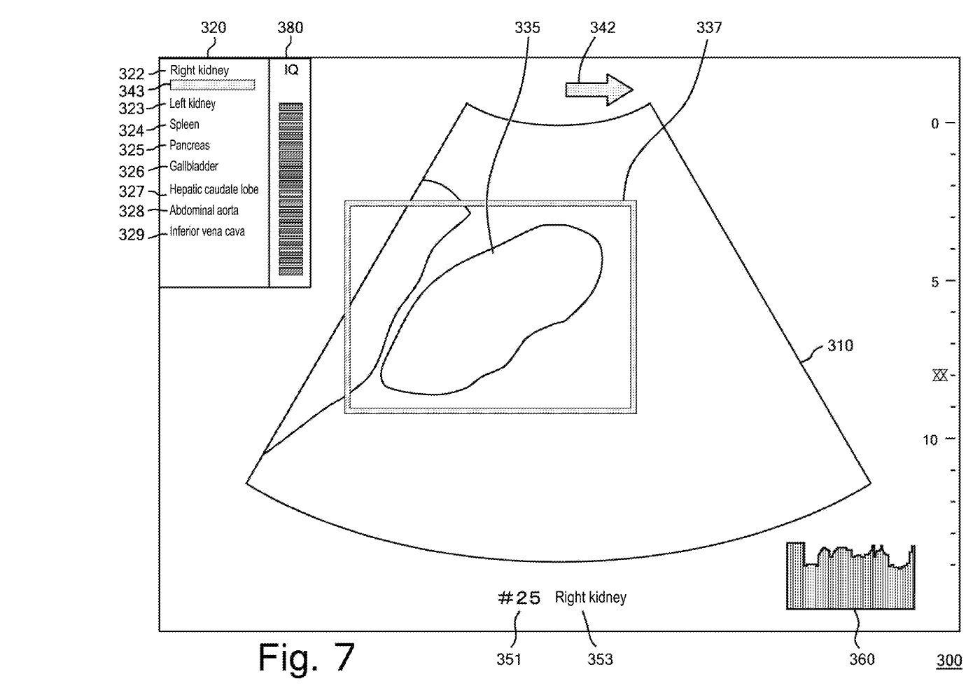

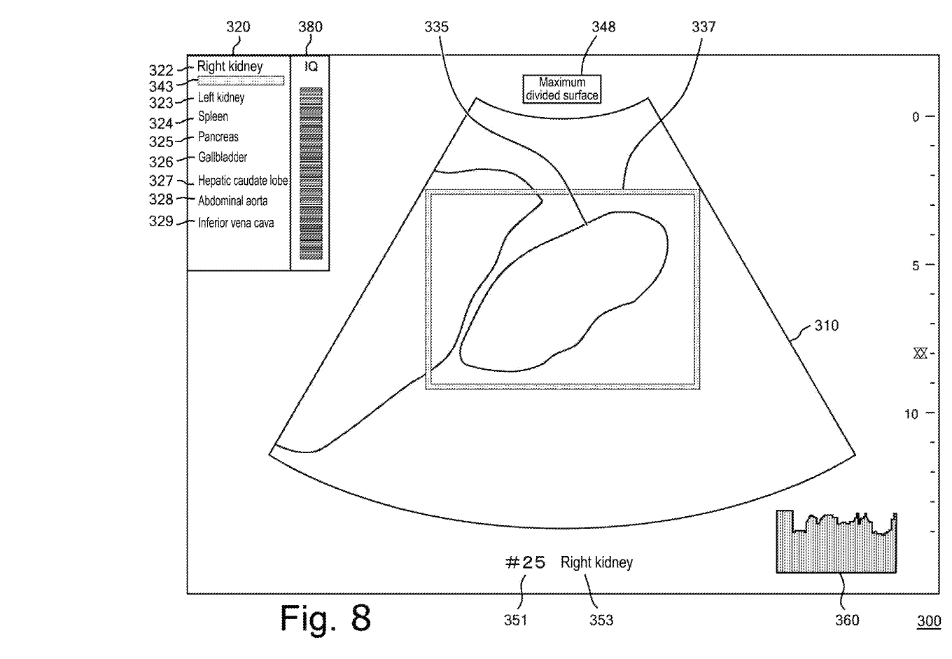

### BRIEF DESCRIPTION OF DRAWINGS

[FIG. 1] A block diagram illustrating a schematic configuration of a system according to an embodiment of the present invention.

[FIG. 2] A block diagram illustrating a configuration of an ultrasound examination device configuring the system illustrated in FIG. 1.

[FIG. 3] A diagram illustrating an image generated by an ultrasound image generation system according to an embodiment of the present invention.

[FIG. 4] A diagram illustrating an image generated by an ultrasound image generation system according to another embodiment of the present invention.

[FIG. 5] A diagram illustrating an image generated by an ultrasound image generation system according to another embodiment of the present invention.

[FIG. 6] An enlarged view of a collection of area indicators.

[FIG. 7] A diagram illustrating an image at a different time point generated by an ultrasound image generation system according to an embodiment of the present invention.

[FIG. 8] A diagram illustrating an image at a different time point generated by an ultrasound image generation system according to an embodiment of the present invention.

### DETAILED DESCRIPTION

Embodiments of the present invention will be described below. Note that the invention claimed in the embodiments described herein is not limited. In particular, in the present disclosure, a medical ultrasound diagnostic system is described as an example. However, the present invention may be applied to an ultrasound examination system, an ultrasound examination device, and an ultrasound probe for the non-destructive examination of buildings, structures, various mechanical devices, and the like.

Embodiments of the present invention will be described below with reference to the drawings. A system 100 illustrated in FIG. 1 is provided with a plurality of ultrasound examination devices 101, 102, and 103, and a server 104. The plurality of ultrasound examination devices 101, 102, and 103 may be communicatively connected to each other via a network 105. In addition, each of the plurality of ultrasound examination devices 101, 102, and 103 may also be communicatively connected to the server 104 via the network 105. A learned model production terminal 107 that produces a learned model may also be communicatively connected to the server 104 via the network 105. The learned model used in the present invention can be transmitted from the learned model production terminal 107 to the plurality of ultrasound examination devices 101, 102, and 103 via the server 104 or without passing through the server 104. In addition, the learned model production terminal 107 can receive training data from the plurality of ultrasound examination devices 101, 102, and 103 via the server 104 or without passing through the server 104. Furthermore, other software associated with the present invention may be downloaded from the server 104 to the plurality of ultrasound examination devices 101, 102, and 103.

The configuration of each of the ultrasound examination devices 101, 102, and 103 is illustrated in FIG. 2 as an ultrasound examination device 200. The ultrasound examination device 200 includes a transmission beamformer 203 for driving (driving) a plurality of vibration elements 201 arranged in an ultrasound probe 202 to generate a pulse ultrasound signal, and a transmitter 204 for emitting the generated pulse ultrasound signal to a subject (not illustrated). The pulse ultrasound signals generate an echo that reflects within the subject and returns to the vibration elements 201. The echo is converted to electrical signals by the vibration elements 201, and these electrical signals are received by a receiver 205. The electric signals representing the received echo, that is, the echo signals are amplified and the like by a required gain in the receiver 205 and then input to a reception beamformer 206, and reception beamforming is performed in the reception beamformer 206. The reception beamformer 206 outputs ultrasound data after reception beamforming.

The reception beamformer 206 may be a hardware beamformer or a software beamformer. When the reception beamformer 206 is a software beamformer, the reception beamformer 206 may include one or a plurality of processors 207, including any one or more of a graphics processing unit (GPU), a microprocessor, a central processing unit (CPU), a digital signal processor (DSP), or other types of processors capable of performing a logical operation. The processor configuring the reception beamformer 206 may be configured by a processor different from the processor 207 to be described later or may be configured by the processor 207. The echo signal before the reception beamforming and the ultrasound data after the reception beamforming are stored in a memory 209.

When echo signals are received, the processor 207 may process the data in real time during a scan session. For the purpose of this disclosure, the term “real time” is defined to include procedures that are performed without any deliberate delay.

In addition, the data may also be temporarily stored in a buffer (not illustrated) during scanning of the ultrasound waves and may be processed in live or off line operations rather than real time. In this disclosure, the term “data” in the present disclosure may be used to refer to one or a plurality of data sets acquired using an ultrasound examination device.

The ultrasound data can be processed by the processor 207 with another or different mode-related module (for example, B-mode, color Doppler, M-mode, color M-mode, spectral Doppler, contrast mode, elastography, TVI, strain, strain rate, and the like) to create ultrasound image data. For example, one or a plurality of modules may generate an ultrasound image, such as B-mode, color Doppler, M-mode, color M-mode, spectral Doppler, contrast mode, elastography, TVI, strain, strain rate, combinations thereof, and the like.

A video processor module may be provided that reads an image frame from the memory while the procedure is being performed on the subject, displaying the image frame in real time. The video processor module may save the image frame in an image memory, and ultrasound images are read from the image memory and displayed on the display 208 (display: display device).

Note that as used in the present specification, the term "image" broadly refers to both visible images and data representing visible images. In addition, the term “data” can include raw data (raw data), which is ultrasound data (an echo signal or a sound ray signal) before a scan conversion operation, and image data, which is data after the scan conversion operation. The processor 207 can further process information obtained by analyzing the ultrasound image and display the information on the display 208 together with the ultrasound image.

When the processor 207 includes a plurality of processors, the plurality of processors may be responsible for the aforementioned processing tasks assigned by the processor 207. For example, the first processor may be used to demodulate and decimate RF signals, while the second processor may be used to further process the data and then display images. In addition, for example, when the reception beamformer 206 is a software beamformer, a processing function thereof may be performed via a single processor or via a plurality of processors.

The display 208 is an LED (Light Emitting Diode) display, an LCD (Liquid Crystal Display), a micro LED display, an organic EL (Electro-Luminescence) display, or the like. The display 208 is not necessarily a single display, and a plurality may be provided. When a plurality of displays are provided, all or many of the displays may be main displays, or one of the displays may be a main display and one or more other displays may be auxiliary displays. The auxiliary display may be, for example, one or more LED elements disposed on a keyboard, ultrasound probe 202, and / or other constituent elements of a user interface 210 described below.

The memory 209 is any known data storing medium. In one example, the ultrasound examination device 200 includes a plurality of memories 209, including a non-transitory storage medium and a transient storage medium as the memory 209. The non-transitory storage medium is, for example, a non-volatile storage medium such as an HDD (Hard Disk Drive: hard disk drive), a ROM (Read Only Memory), or the like. The non-transitory storage medium may include a portable storage medium such as a CD (Compact Disk), DVD (Digital Versatile Disk), a Blu-ray disc (Blu-ray Disc (TM)), or the like. A program executed by the processor 207 is stored in the non-transitory storage medium. In addition, the non-transitory storage medium also stores a protocol, a learning model, image data, and the like necessary for implementing the present invention. The transitory storage medium is a volatile storage medium such as a RAM (Random Access Memory). All of these may be stored in the same memory 209, or at least one of these may be stored in a different memory 209. In addition, the memory 209 may also be a plurality of data storage mediums deployed on a cloud.

A user interface 210 may accept input from an operator. For example, the user interface 210 accepts commands and information input from an operator. The user interface 210 is configured by including a keyboard (keyboard), hard keys (hard key), soft keys, and the like. The user interface 210 may include various input devices such as pointing devices such as a mouse, a touch panel, a pen tablet, a touch pad, a trackball, and a joystick, as well as eye tracking and voice input devices.

A speaker 211 is controlled by the processor 207 to output sound. In one example, the speaker 211 outputs sound based on a signal input from the processor 207.

As illustrated in FIG. 1, a communication interface (not illustrated) may be provided to enable the ultrasound examination devices 101 to 103 (each corresponding to the ultrasound examination device 200 in FIG. 2) to communicate with the server 104 or the like. However, in other embodiments, the ultrasound examination device 200 may operate in a stand-alone state to implement the present invention. In this case, the communication interface is not required.

FIG. 3 is a diagram illustrating an image 300 generated by the ultrasound image generation system according to an embodiment of the present invention. In this embodiment, a navigation function as described in Patent Document 2 is used. The navigation function shows organs that must be measured in a pre-registered order so that no omission occurs in an image that must be acquired for diagnosis, and navigates (guides) an operator so that an examination is performed at a correct position. In this example, the imaging subject is a human body and the structure to be examined is an organ of the human body or a part thereof.

For example, when it is necessary to perform an ultrasound examination on the entire target site evenly for the purpose of a health diagnosis or the like, the regions to be examined are registered in order so that the examination is performed according to a predetermined procedure in order to prevent leakage. The operator can proceed with the operation by referring to the displayed comments or reference images. The operator examines a necessary region according to the instructions and leaves an image record. The recorded image is used as evidence of thorough examination and for reporting the presence or absence of detected abnormalities. At this time, it is required to draw the organ designated in each step to the center of the screen and in the maximum divided surface as much as possible and record the image.

An examination step display box 320 is displayed at the upper left of FIG. 3. In the examination step display box 320, examination structure names 321 to 329 of the examination subject in each step for the navigation are displayed. The examination is scheduled to be performed in the order of the examination structure names 321 to 329 displayed in the examination step display box 320, and the navigation is performed in this order. In the example of FIG. 3, a state is illustrated in which step 321 of the examination of a liver 331 has ended and step 322 of the examination of a right kidney 335 has started. In the embodiments of the invention, the right kidney 335 will be mainly described, but other target organs can be similarly processed. In addition, as described above, when applied to an industrial ultrasound examination device, the target organ may be replaced with an internal structure such as a pipe or a valve and the present invention may be implemented.

Referring to FIG. 3, a current examination step number 351 and a current examination step name 353 are displayed in a lower portion of a screen 300 of the display 208. In this example, the current examination step number 351 is 25, and the right kidney, which is the structure (organ) to be examined, is registered as the step name of the current examination step name 353. The type of organ to be selected as the examination item and the order of the examination is customizable, and the way of setting the examination step name 353 is also customizable. In certain embodiments of the present invention, in addition to customizing which organs are in which order, routines may be implemented to prompt a comment at a specific step (for example, existence of fatty liver, or cholecystectomy) to tell which mode to switch to at which step (for example, Doppler mode, and the like). In each examination step, comments and annotations can be automatically, semi-automatically, or manually recorded in the storage device (memory) 209 in addition to the cross-sectional image of the target organ. The comment or annotation may be a change in the lesion, a rate of change, a name of a suspected lesion, a determination classification thereof, a stage, a congenital deformity, a width of a bile duct or an aortic aneurysm, or a figure such as an arrow or a circular enclosure disposed at a specific position. The comment or the annotation is displayed in a manner in which the operator can correct the comment or the annotation based on automatic recognition by AI, and after confirming the content, the operator can store the comment or the annotation with or without correcting the content.

The ending of the current step and the switching to the next step, that is, the switching of the target organ, can be performed in response to various events. In a specific embodiment of the present invention, the next target organ may be moved to in response to the occurrence of an event of saving an image of the current target organ. After moving to the next target organ, only the next target organ can be tracked. In addition, for example, the operator selects an examination step 324 of a spleen displayed in the examination step display box 320 and instructs the software to set such as the current target organ, whereby the examination step 323 of the left kidney can be skipped and the examination step 324 of the spleen and the subsequent steps can be performed. In a specific embodiment, when the target organ is set to a specific organ, even if other organs are recognized by AI, other organs or structures that are not the target organ are ignored, and only the target organ is tracked. In a specific embodiment of the present invention, the names of all organs and structures detected in the B-mode image 310 and their detection accuracy are displayed.

In the example of FIG. 3, the ultrasound probe 202 is arranged on the subject, the ultrasound probe 202 receives echo signals from a portion including a liver 331 and a right kidney 335 of the subject, and a B-mode image 310 is generated. At this point, the B-mode image 310 is analyzed. As a result of this analysis, the presence of the liver 331 and the right kidney 335 of the subject in the B-mode image 310 is specified. In the example of FIG. 3, a frame (bounding box) 333 surrounding the liver 331 is illustrated in, for example, dark gray, and a frame 337 surrounding the right kidney 335 is illustrated in, for example, light green so that the operator can recognize a state in which step 321 of the examination of the liver 331 is completed and step 322 of the examination of the right kidney 335 is started. Frames 333 and 337 indicate that these structures are recognized as specific structures. These frames need not be rectangular, but may be circular, elliptical, polygonal, or the like. In addition, as illustrated in FIG. 4, the recognized structure can be shown to the operator by segmenting the recognized structure and superimposing FIGS. 332 and 336 obtained by segmenting and tracing the recognized structure on the B-mode image in a translucent and / or blinking manner. In FIG. 4, the FIGS. 332 and 336 are drawn with shapes and sizes completely matching the cross-sections of the liver 331 and the right kidney 335, but the sizes may be increased or decreased by about 1 to 10%. Alternatively, the central portion may be removed to form an annular base along the contour.

In a specific embodiment, the AI learning model determines whether the organ set in each step for the navigation software is depicted on the screen. The neural network of the AI learning model may be various types of neural networks such as deep learning, DeepDream, RNN, CNN, diffusion, GAN, or the like. The AI learning model may detect which type of organ is included in the ultrasound image. In addition, the probability (accuracy) that the detected organ is the organ can be obtained. The detected organ and the accuracy can be displayed in association with the image of the organ.

In a specific embodiment, the AI detects the region or shape of one or more organs included in the ultrasound image, and confirms whether the designated target organ is included in each step. If the current target organ is included, an instruction to move the probe in a direction to draw the region as close to the center of the screen as possible is output. In the example of FIG. 3, an arrow 340 prompting an operation is displayed on the screen so that the frame 337 surrounding the right kidney 335 is drawn at the center of the B-mode image 310. The arrow 340 is drawn in the same color as the frame 337 surrounding the right kidney 335, and the operator can easily understand that the arrow is an instruction for the right kidney 335. The length of the arrow 340 can be changed depending on the required moving distance. That is, when the necessary movement distance is long, the arrow 340 is also drawn long accordingly, and when the necessary movement distance is short, the arrow 340 is also drawn short accordingly. The necessary moving distance can be expressed by the thickness, brightness, color, or the like of the arrow 340 instead of the length of the arrow.

The position of the predetermined structure exemplified by the right kidney 335 in the ultrasound image can be specified by various methods. For example, the intersection of the diagonal lines of the frame 337 illustrated in FIG. 3 can be set as the position of the center of the right kidney 335. In addition, the position of the center of the right kidney 335 can be the average of the positions of the pixels included in the FIG. 336 obtained by tracing illustrated in FIG. 4. In the former case, although the desired position is not necessarily accurate, the amount of calculation is small and the calculation can be completed quickly. In the latter case, the amount of calculation is large, but a more accurate position can be specified. In addition, in a specific embodiment, when a lesion is detected in the right kidney 335, a point considering the position of the lesion may be the position of the center of the right kidney 335. For example, the midpoint between the position of the center of the right kidney 335 not considering the lesion and the position of the center of the region occupied by the lesion may be set as the position of the center of the right kidney 335 having the lesion. In other embodiments, the presence or absence of a lesion in the right kidney 335 is not considered and the location of the center of the right kidney 335 is calculated.

The guide information instructing the operator operating the ultrasound probe 202 to move the ultrasound probe to a position where an ultrasound image can be obtained in which the predetermined structure is displayed more favorably is embodied by an arrow 340 in FIGS. 3 and 4. However, the guide information may be embodied in various other ways. In the example of FIG. 5, a zebra line 344 is displayed to prompt the operator to perform an operation to shift the B-mode image 310 itself to the right. The zebra line 344 may be fixed, but the zebra line 344 may be moved or made to blink by an animation. The guide information may be characters, and for example, character information "(ultrasound probe 202) 4cm to the right" can be displayed on the screen 300. Furthermore, LEDs may be disposed on both sides of the ultrasound probe 202 and / or the keyboard of the user interface 210, and the direction to be moved may be indicated by illuminating the LEDs. This is not limited to the arrow 340, the zebra line 344, and the LED described here, and the moving direction guide display can be embodied in various aspects. For example, the moving direction can be guided by voice, or a region of the patient to be brought into contact with the ultrasound probe 202 (for example, a position between a right 7th rib and an 8th rib) can be indicated to the examiner by voice or animation.

As described above, in a specific embodiment, the AI detects the region or shape of one or more organs included in the ultrasound image, and confirms whether the designated target organ is included in each step. The area in the ultrasound image of the predetermined structure, exemplified by the right kidney 335 in FIG. 3, can be calculated in a variety of ways. For example, the area of the frame 337 illustrated in FIG. 3 can be approximated as the drawing area of the right kidney 335. In addition, for example, the number of pixels included in the FIG. 336 obtained by tracing illustrated in FIG. 4 can be set as the area. In the former case, although the desired area is not necessarily accurate, the amount of calculation is small and the calculation can be completed at high speed. In the latter case, the amount of calculation is large, but a more accurate area can be specified.

The calculated area of the right kidney 335 is displayed on the screen 300 as a set of area indicators 360. FIG. 6 is an enlarged view of the set of area indicators 360. In the example of this figure, a rightmost area indicator 361 is the most recent, and a leftmost area indicator 365 is the oldest. The area indicators 361 to 365 are generated at a predetermined sampling rate. The area indicators 361 to 365 can be set to different colors for each type of organ, or can be set to the same color for all organs. The predetermined sampling rate is preferably equal to the frame rate of the image. When a new area indicator is generated, the existing area indicators 361 to 365 shift to the right, and the area indicator 365 disappears from the screen 300. A horizontal axis 368 is time and a vertical axis 369 represents the area of the target organ. It is possible to transform the vertical axis 369 to represent time and the horizontal axis 368 to represent the area of the target organ, or to change the time axis to be reversed (that is, left shift to right shift). These changes may also be customizable by the operator. By displaying a plurality of area indicators on the display device at the same time, the operator can confirm how the area in which the target organ is depicted changes with time. That is, the set of area indicators 360 is output in a manner that allows changes in the area of the target organ over time at a plurality of time points to be recognized.

In the example of FIG. 6, the area indicator is embodied in the form of a bar graph, with the area of the target organ reflected in the length attribute of the bar graph. The area indicator can be modified and implemented in various ways. For example, the bar graph of FIG. 6 may be a line graph or a simple plot. In this case, the area of the target organ is reflected in an attribute such as the height (coordinate position) of the line graph or the plot. In addition, image items having the same shape may be arranged in the vertical direction or the horizontal direction, and a green color may be set for when the area of the target organ becomes larger, and a red color may be set for when the area of the target organ becomes smaller, or a higher luminance may be set when the area of the target organ becomes larger, and a lower luminance may be set when the area of the target organ becomes smaller. Furthermore, the area indicator may be represented by an animation. For example, as the area of the target organ increases, the label, symbols, markers, and the like representing that organ may become active or exaggerated. In such a case, the area of the target organ is reflected in the attribute of the shape of the area indicator. The area of the target organ may be displayed simply as a numerical value. The examiner can refer to the change in the area indicator over time and record the maximum divided surface as much as possible. The examiner can refer to the change in the area indicator with time, move the probe (in the direction perpendicular to the cross section), and store the image at the position where the cross-sectional area becomes larger.

Returning to FIG. 3, to the right of the examination step display box 320, an image quality (IQ: image quality) gauge 380 is displayed. In this example, the image quality gauge 380 is configured such that the number of illuminated squares of the image quality gauge 380 increases as the image quality (IQ: image quality) score is higher, and the number of illuminated squares of the image quality gauge 380 decreases as the image quality score is lower. The image quality score is calculated according to the area of the target organ, the position of the target organ, and the degree of noise and / or artifact included in the target organ. As is well known to those skilled in the art, noise generated in an ultrasound image includes that which is caused by EMI, a power supply, or the like. As is well known to those skilled in the art, artifacts generated in an ultrasound image are generated due to various factors such as poor contact with the probe, the presence of a calcified viscera or organ, metal present in the body, and bone fragments, and appear in the image as acoustic shadows, reverberations, blurring, or the like.

Each score that underlies the image quality score can be calculated using a variety of functions. For example, the area score can be calculated based on "current area ÷ maximum area observed during a set period of time". The score of the position can be obtained by substituting the distance from the center into a parabolic function having a y-intercept of 1 and a convex upward. The noise and / or artifact score may be calculated based on "(area of entire image - area of noise or artifact) ÷ area of entire image".

The image quality score may be calculated, for example, by taking the weighted average of the area score, the position score, and the noise and / or artifact score. In a specific embodiment, the noise and / or artifact score may be excluded from the calculation of the image quality score, and the image quality score may be calculated from the area score and the position score alone. Each score may be a gauge indicating a respective level for each item. In addition, for example, a gauge may be displayed that combines the position score and the noise and / or artifact score, but excludes the area score, and other combinations of gauges are also possible. The image quality gauge 380 can change the color, such as red for a low score, blue for a good score, and yellow for a middle score, or can change the brightness. The image quality gauge 380 may have another shape such as an annular shape instead of a linear shape.

FIG. 7 is a diagram illustrating an image at another time point generated by the ultrasound image generation system according to an embodiment of the present invention. As illustrated in FIG. 7, the frame 337 surrounding the right kidney 335 has shifted to the left. An arrow 342 prompting an operation is displayed on the screen so that the frame 337 surrounding the right kidney 335 is drawn at the center of the B-mode image 310. The image quality score indicated by the image quality gauge 380 is higher than that illustrated in FIG. 3 and the like. The arrow 342 is also drawn in the same color as the frame 337 surrounding the right kidney 335, and the operator can easily understand that the arrow is an indication of the right kidney 335. The arrow 342 is shorter than the arrow 340 in FIG. 3. In this example, the ultrasound probe 202 is moved according to the guide information 340 and 344 in FIGS. 3 to 5, but is in a state of being moved too much due to an erroneous operation, and the arrow 342 in FIG. 7 indicates a direction opposite to the arrow 340 in FIG. 3.

The image quality gauge may also be displayed in association with the step for examination of the target organ. In the example of FIG. 7, the image quality gauge 343 of the right kidney is displayed below the examination step 322 of the right kidney. When the image quality gauge is displayed in association with the step for the examination of the target organ, there is an advantage that it is possible to confirm which organ is currently depicted in the B-mode image 310. In addition, as in the example of FIG. 3, there is an advantage that the image quality of the target organ in the previous examination step and the current examination step can be confirmed in a state where step 321 of the examination of the liver 331 is completed and step 322 of the examination of the right kidney 335 is started.

FIG. 8 is a diagram illustrating an image at another time point generated by the ultrasound image generation system according to an embodiment of the present invention. As illustrated in FIG. 8, the frame 337 surrounding the right kidney 335 is depicted at the center of the B-mode image 310. The image quality score indicated by the image quality gauge 380 is higher than that indicated in FIG. 7. In a specific embodiment of the present invention, an image saving process event is automatically initiated when the image quality score exceeds a predetermined value. In addition, when it is recognized by AI that the target organ is depicted in the maximum divided surface, a display 348 indicating to the examiner that the target organ is depicted in the maximum divided surface is displayed on the screen 300 (display of the maximum divided surface 348). In another embodiment, the cross-sectional area is not estimated by AI, and the maximum divided surface is specified by estimating a highest maximum value using an area change curve obtained by moving the ultrasound probe 202. In a specific embodiment of the present invention, the current examination step is automatically terminated and the examination step for the next target organ is automatically started in response to the image saving process event ending. In another specific embodiment of the present invention, in response to the image saving process event ending, a display is output prompting the operator to end the current examination step and to start the examination step for the next target organ. The operator can instruct the software to move the examination step to the next step accordingly. In the case of FIG. 8, the next examination step is the examination step 323 of the left kidney. In this example, the left kidney is not depicted in the B-mode image 310. Therefore, the moving direction guide display indicating the moving direction such as the arrow 340, the zebra line 344, or the LED is not output, and for example, the specific position of the patient to be in contact with the ultrasound probe 202 or what kind of instruction will be given to the patient (moving the body from the posture of lifting the right side of the waist to the posture of lifting the left side of the waist) can be indicated to the examiner by a tutorial using a sound and / or an animation.

In a specific embodiment, examination steps may be added automatically. For example, when a malignant tumor is detected in the right kidney 335, the region recognized as the malignant tumor becomes the next target organ, and the examination step is executed. In this examination step, navigation is performed to obtain the maximum divided surface of the region recognized as a malignant tumor. A message is output to the examiner indicating that a special examination step has been added.

The image saving process event can be executed in various ways. For example, as a background process that is not recognized by the examiner, a plurality of images whose image quality scores exceed a predetermined value can be saved. The examiner can select one or more images from the plurality of images as necessary and set the selected images as images to be saved last. In another embodiment, at a time point where the image quality score exceeds a predetermined value, the B-mode image 310 is frozen, and a message is output to prompt the examiner to save the frozen B-mode image 310. The examiner can save the B-mode image 310 in response to this. When the B-mode image 310 is frozen, the update of the B-mode image 310 is stopped, and the B-mode image is saved as necessary by the examiner, and the frozen state is maintained until the freeze is released or the power is turned off. In another embodiment, the B-mode image 310 is kept frozen for a predetermined time. The predetermined time may be customizable.

Note that the invention is not limited to the present embodiment, and various modifications are possible without departing from the essence of the invention.

### DESCRIPTION OF REFERENCE NUMERALS

100: System

101, 102, 103: Ultrasound examination device

104: Server

105: Network

107: Trained model production terminal

200: Ultrasound examination device

201: Vibration elements

202: Ultrasonic probe

203: Transmission beamformer

204: Transmitter

205: Receiver

206: Reception beamformer

207: Processor

208: Display

209: Memory

210: User interface

211: Speaker

300: Image

310: B-mode image

320: Examination step display box

321: First examination structure name

322: Second examination structure name

323 to 329: Other examination structure name

331: First examination structure

332: Figure corresponding to first examination structure

333, 337: Frame

335: Second examination structure

336: Figure corresponding to second examination structure

340, 342: Arrow

341, 343: Image quality gauge

344: Zebra line

348: Display of maximum divided surface

351: Current examination step number

353: Current examination step name

360: Set of area indicators

361 to 365: Area indicator

368: Horizontal axis

369: Vertical axis

380: Image quality gauge

What is claimed is:

1. An ultrasound image generation system for displaying an ultrasound image on a display device, the ultrasound image generation system comprising:
a memory storing instructions;
a processor configured to execute the instructions to:
analyze a first ultrasound image of an ultrasound probe at a first time to specify a predetermined structure within an imaging subject;
specify a first position in the first ultrasound image of the predetermined structure;
output a first area indicator having an attribute corresponding to a first area in the first ultrasound image of the predetermined structure;
output guide information for instructing an operator who operates the ultrasound probe to move the ultrasound probe to a position where an ultrasound image in which the predetermined structure is displayed more favorably can be obtained, based on the identified first position;
output a second area indicator having an attribute corresponding to a second area in a second ultrasound image at a second time point of the predetermined structure in a manner that a change over time with respect to the first area indicator is recognizable.

2. The ultrasound image generation system according to claim 1, comprising: the display device and the ultrasound probe,
wherein the first area indicator and the second area indicator are displayed simultaneously on the display device; and
wherein the attribute includes any of a shape, coordinate position, luminance, and color of the first area indicator and / or the second area indicator.

3. The ultrasound image generation system according to claim 1, wherein the processor is further configured to execute the instructions to output, to the display device, a moving direction guide display corresponding to a direction of the second ultrasound probe position with respect to the first ultrasound probe position as at least a part of the guide information.

4. The ultrasound image generation system according to claim 1, wherein the processor is further configured to execute the instructions to output, to a speaker of the ultrasound image generation system, at least a part of the guide information, a moving direction guide voice corresponding to a direction of the second ultrasound probe position with respect to the first ultrasound probe position.

5. The ultrasound image generation system according to claim 1, wherein the memory stores a learned model for specifying the predetermined structure in the imaging subject.

6. The ultrasound image generation system according to claim 1, wherein the imaging subject is a human body, and the structure is an organ of the human body or a portion thereof.

7. The ultrasound image generation system according to claim 1,
wherein the processor is further configured to execute the instructions to:
identify a workflow defining an order in which at least a first structure and a second structure in the imaging subject are to be imaged;
wherein based on the workflow being in a process of imaging the first structure, the analyzing the first ultrasound image to identify the predetermined structure in the imaging subject comprises:
analyzing the first ultrasound image to identify the first structure and the second structure in the imaging subject;
ignoring the second structure while making the first structure the predetermined structure.

8. The ultrasound image generation system according to claim 7, wherein the workflow is customizable, the workflow includes a change in a scan mode of the ultrasound image generation system, and the structure is an organ of the human body or a portion thereof.

9. The ultrasound image generation system according to claim 1, wherein the processor is further configured to execute the instructions to:
confirm whether a second position and the second area of the predetermined structure in the second ultrasound image satisfy a predetermined criterion;
based on the second position and the second area satisfy the predetermined criterion, automatically save the second ultrasound image; or
maintain the display of the second ultrasound image on the display device for a predetermined time interval or longer.

10. The ultrasound image generation system according to claim 1, wherein the processor is further configured to execute the instructions to:
confirm whether a second image quality, a second position, and the second area of the predetermined structure in the second ultrasound image satisfy a predetermined criterion;
based on the second image quality, the second position, and the second area satisfy the predetermined criterion, automatically save the second ultrasound image; or
maintain the display of the second ultrasound image on the display device for a predetermined time interval or longer.

11. The ultrasound image generation system according to claim 8, wherein the first area is an area of the predetermined structure or an area of a figure corresponding to the predetermined structure.

12. The ultrasound image generation system according to claim 11, wherein the figure corresponding to the predetermined structure is a rectangle or a polygon obtained by segmenting the predetermined structure.

13. The ultrasound image generation system according to claim 7,
wherein the processor is further configured to execute the instructions to calculate a second score based on an area, a position, and an image quality of the predetermined structure in the second ultrasound image;
display a second score indicator corresponding to the second score on the display device;
display the second score indicator in a first color and /or a first brightness based on the second score indicating a value equal to or greater than a predetermined value; and
display the second score indicator in a second color and /or a second brightness based on the second score indicating a value less than a predetermined value.

14. The ultrasound image generation system according to claim 13, wherein the image quality includes a noise and /or artifact of the predetermined structure.

15. The ultrasound image generation system according to claim 13, wherein the processor is further configured to execute the instructions to automatically save the second ultrasound image based on the second score indicating a value equal to or greater than the predetermined value.

16. The ultrasound image generation system according to claim 13,
wherein the processor is further configured to execute the instructions to continue displaying the second ultrasound image as a still image on the display device when the second score indicates a value equal to or greater than the predetermined value; or
generate an output for prompting an operator to perform an operation of storing the second ultrasound image as a still image in a storage device when the second score indicates a value equal to or greater than a predetermined value.

17. The ultrasound image generation system according to claim 16, wherein the imaging subject is a patient, and the still image is stored in association with patient identification information associated with the patient and step identification information for identifying a step in the workflow, and is referred to at the time of an ultrasound examination performed on the patient thereafter.

18. The ultrasound image generation system according to claim 1, wherein the first area indicator and the second area indicator are displayed along a time axis.

19. The ultrasound image generation system according to claim 1, wherein the first area indicator and the second area indicator are generated and displayed at a predetermined sampling interval.

20. A non-transitory computer readable medium storing instructions that, when executed by a processor, cause the processor to:
analyze a first ultrasound image of an ultrasound probe at a first time to identify a predetermined structure within an imaging subject;
identify a first position in the first ultrasound image of the predetermined structure;
output a first area indicator having an attribute corresponding to a first area in the first ultrasound image of the predetermined structure;
output guide information for instructing an operator who operates an ultrasound probe to move the ultrasound probe to a position where an ultrasound image in which the predetermined structure is displayed more favorably can be obtained, based on the specified first position;
output a second area indicator having an attribute corresponding to a second area in a second ultrasound image at a second time point of the predetermined structure in a manner that a change over time with respect to the first area indicator is recognizable.
